(19) United States
(12) Patent Application Publication
(10) Pub. No.: US 2023/0078019 A1
(43) Pub. Date: Mar. 16, 2023

(54) ENDOTHERMIC COMPRESSION SYSTEMS AND METHODS

(71) Applicant: Merit Medical Systems, Inc., South Jordan, UT (US)

(72) Inventors: Jacob Munger, Herriman, UT (US); Damian Lynch, Guilford, CT (US); Rob Fredericks, Salt Lake City, UT (US); Aaron Hopkinson, Herriman, UT (US); Alan Vawdrey, Riverton, UT (US); Jordan Peterson, West Jordan, UT (US)

(21) Appl. No.: 17/931,327

(22) Filed: Sep. 12, 2022

Related U.S. Application Data
(60) Provisional application No. 63/244,122, filed on Sep. 14, 2021.

(51) Int. Cl. A61F 7/02

(52) U.S. Cl. CPC A61F 7/02; A61F 2007/0228; A61F 2007/0056

(57) ABSTRACT

Devices and systems for providing cooling and compression to a site needing hemostasis can include a compression member having a component that can be inflated with one or more fluids, including a coolant solution. Cooling can be initiated and maintained by securing such a device to the site and optionally equipping the device with the coolant solution. A system can include materials for equipping the device with a coolant solution.

### RELATED APPLICATIONS

This application claims priority to U.S. Provisional Patent Application No. 63/244,122, filed Sep. 14, 2021, and titled ENDOTHERMIC COMPRESSION SYSTEMS AND METHODS, which is incorporated herein by reference in its entirety.

### FIELD OF THE INVENTION

The present disclosure relates generally to medical devices used to provide compression and cooling to a vascular puncture or wound site as well as related systems and methods. More particularly, some embodiments of the present disclosure relate to endothermic compression devices, systems, and methods used to provide compression and cooling at a surgical site, including surgical sites located on the torso.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A illustrates a use of an endothermic compression device in accordance with one embodiment.

FIG. 1B is a view of the underside of a component of the endothermic compression device shown in FIG. 1A.

FIG. 1C is a view of the top of a component of the endothermic compression device shown in FIG. 1A.

FIG. 1D a top partial view of the endothermic compression device shown in FIG. 1A.

FIG. 2 illustrates a use of an endothermic compression device in accordance with one embodiment.

FIG. 3A is a top view of an endothermic compression device in accordance with one embodiment.

FIG. 3B is a view of the underside of a component of the endothermic compression device shown in FIG. 3A.

FIG. 4A illustrates a use of a system in accordance with one embodiment.

FIG. 4B illustrates a stage of a function of a component of the system shown in FIG. 4A.

FIG. 4C illustrates a subsequent stage of the function of the component of the system shown in FIG. 4B.

FIG. 4D illustrates another subsequent stage of the function of the component of the system shown in FIG. 4B.

FIG. 5 is a cross-section view of a component of an endothermic compression device in accordance with another embodiment.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical and fluidic interaction. Thus, two components may be coupled to each other even though they are not in direct contact with each other.

The phrase “fluid communication” is used in its ordinary sense and is broad enough to refer to arrangements in which a fluid (e.g., a gas or a liquid) can flow from one element to another element when the elements are in fluid communication with each other. Relatedly, “fluidly connect” is used herein to refer to an action or process of establishing a connection that provides fluid communication.

The term “fluid” is used in its broadest sense, to refer to any fluid, including both liquids and gases as well as solutions, compounds, suspensions, etc., which generally behave as fluids.

The term “compression” is used to define a compressive force or pressure applied to a portion of a patient over an area. The compression level may correlate to a pressure within an inflatable component of a compression device. The compression level may also correlate to a volumetric size or shape of an inflatable component. The compression level may also correlate to a downward force of the compression device.

The term “inflation” refers to the condition of an expandable fluid container. An increase of inflation correlates to an increase in fluid content within the container or to the volumetric size of the container. The inflation fluid may be compressible or non-compressible. The inflation level may or may not correlate to an internal pressure.

In some instances, surgery may cause some internal and/or external bleeding at or near the surgical site. This may lead to unwanted blood loss, development of hematoma, or other undesired outcomes. Thus, control of bleeding and/or establishing hemostasis may be part of a variety of treatments and procedures. In some instances, application of compression and/or cooling at an incision site may reduce the amount of bleeding and establish hemostasis after surgery. These measures can each reduce the diameter of blood vessels at and around the site, where such reduction is accomplished mechanically by compression, and/or via vasoconstriction induced by cooling.

The placement of pacing devices, such as a pacemaker or defibrillator, within a patient may include surgical creation of a pocket to receive the pacing device. Such a pocket may be formed in the tissue under the patient's skin on the torso of the patient, for example on the upper part of the patient's chest near the pectoral muscle and/or collarbone. In some instances, the external incision associated with creation of the pocket may be between one and three inches long. Creation of the pocket may cause internal and/or external bleeding and, in some instances, application of compression and/or cooling to the surgical site may reduce or otherwise aid in controlling the bleeding.

In some therapies, a pacing device may be located adjacent or near the subclavian or brachial vein and may include one or more pacemaker leads extending to the heart through the vasculature, such as through one of the subclavian or brachial veins. Again, the pacing device may also be disposed within a pocket in the tissue of the patient. Surgical incisions and tissue removal may thus create a surgical or incision site, which may also be referred to herein as the compression site, as application of compression with cooling may aid in controlling bleeding of the surgical or incision site after surgery.

Certain embodiments described herein facilitate the application of cooling with or without pressure to promote hemostasis at a torso surgical site. However, the present disclosure is not so limited; the application of cooling to promote hemostasis may be applied at access sites involving arteries and veins in various vasculature access points in a patient's body. Therefore, though specific examples in the disclosure below may refer to applying cooling to a torso surgical site, the embodiments of the present disclosure may be directed to other sites, such a patient's arm, wrist, hand, leg, or foot. For example, the embodiments of the present disclosure may be configured to promote hemostasis at a femoral artery access site, or at a radial artery access site.

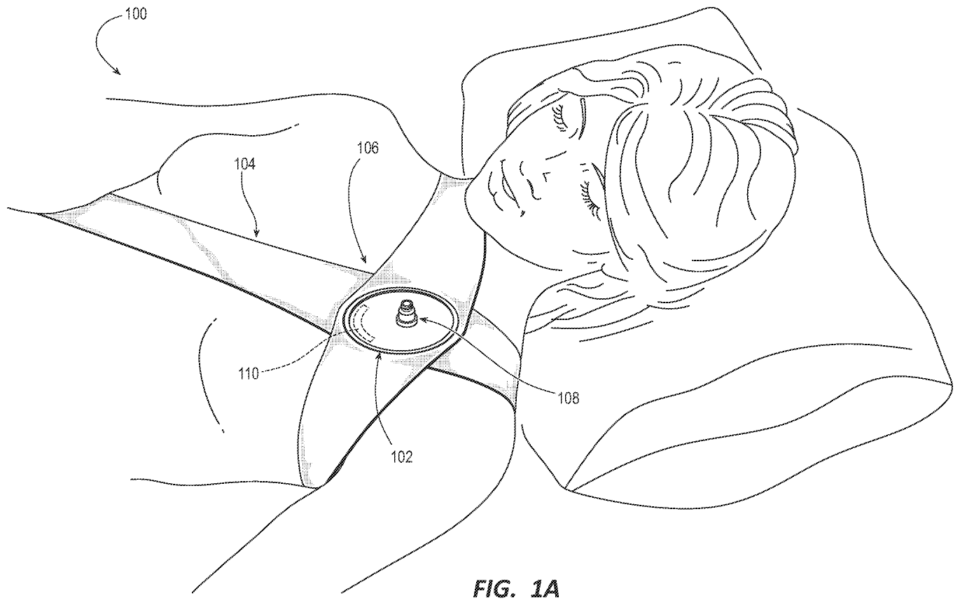

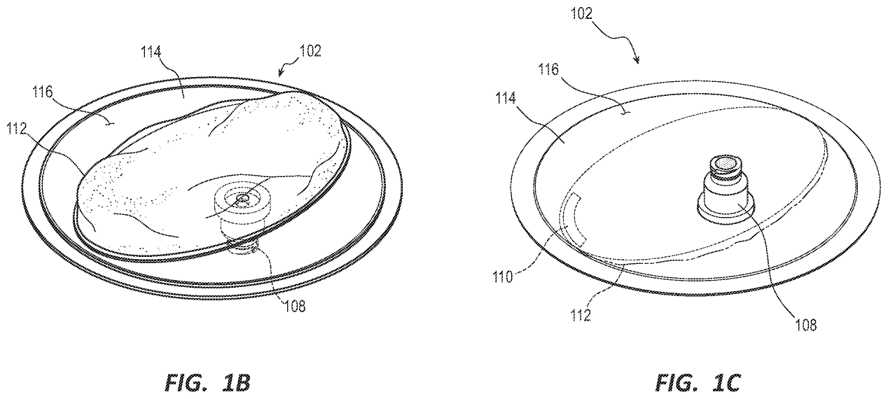

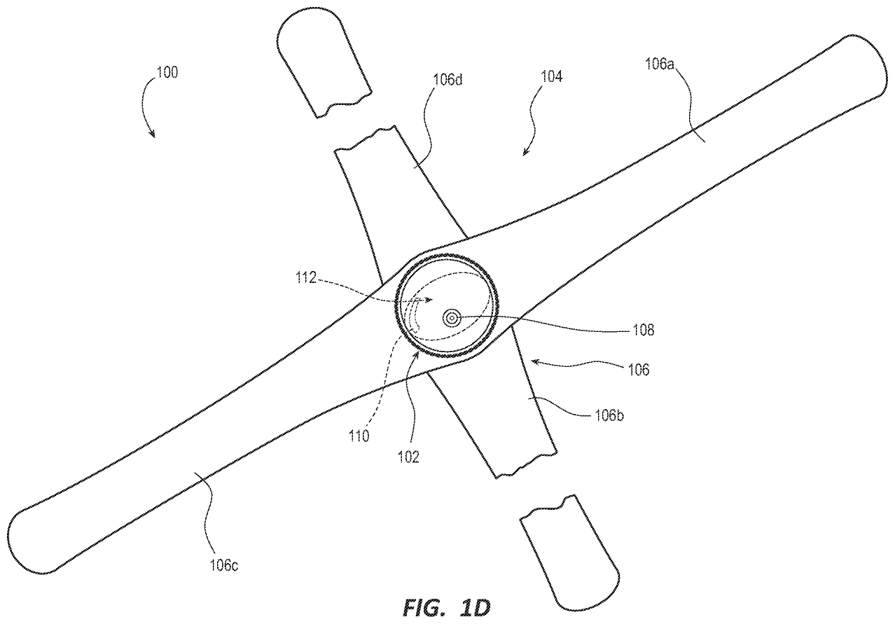

FIG. 1A is an illustration of an embodiment of an endothermic compression 100 device in use on a patient. The compression device 100 may be configured to provide both a variable compressive pressure and cooling to a compression site. The compression device 100 may comprise a compression member 102 and a securement system 104. The securement system 104 may be coupled to the compression member 102 and be configured to facilitate attachment of the compression member 102 to a patient so as to be situated over a compression site. The securement system 104 may be configured to provide positional stability of the compression member 102 over the compression site. The securement system 104 may comprise a plurality of straps 106 that may be coupled the compression member 102. While the securement system 104 in FIG. 1A is shown in use on a torso compression site, securement systems configured for attachment to other body regions such as the wrist or a thigh are also encompassed by the present disclosure. The compression member 102 may comprise a valve 108 as further described below. The compression member 102 may also comprise a temperature indicator 110 as further described below.

FIG. 1B and FIG. 1C illustrate various views of the compression member 102 of the compression device 100 in which the securement system 104 is not shown. As shown in FIG. 1B and FIG. 1C, the compression member 102 may comprise an inflatable bladder 112 and a valve 108. FIG. 1B is a bottom view of the compression member 102 and FIG. 1C is a top view of the compression member 102. The inflatable bladder 112 may be disposed on the bottom or underside of the compression member 102 so as to be disposed adjacent the skin of a patient when placed over the compression site. The compression member 102 can comprise a top plate 114 configured to provide a support for the inflatable bladder 112. The top plate 114 may be rigid. The top plate 114 may also be semi-flexible or flexible so as to conform to the anatomy of a patient upon securement and/or flex as the bladder is inflated. The top plate 114 may comprise a substantially flat plate, and/or may comprise flat, curved, convex, or concave portions. Further, the top plate 114 may be symmetrical or non-symmetrical.

The compression member 102 may include a window 116 to facilitate visual observation of the compression site when the compression member 102 is in place. As shown in FIG. 1C, the top plate 114 may be transparent or translucent so as to define a window 116 so that the compression site and/or the inflatable bladder 112 may be visually observed through the top plate 114. Such visual observation may facilitate alignment of the compression member 102 with the compression site and assessment of hemostasis during treatment.

The inflatable bladder 112 may define a fluid-tight interior that allows it to be inflated by introduction of a fluid into the interior. The inflatable bladder 112 may comprise a material that extends around the entire interior, or the bladder material may be joined to the underside of the top plate 114 so as to define the interior. The inflatable bladder 112 may be configured so as to provide compression to a compression site when the inflatable bladder 112 is at least partially inflated. The inflatable bladder 112 may comprise a flat sheet or a pre-formed three-dimensional shape. The inflatable bladder 112 may be flexible and non-stretchable or flexible and stretchable. The inflatable bladder 112 may be transparent or translucent to facilitate visible observation of a compression site through both the window 116 and the inflatable bladder 112.

In addition to compression, the inflatable bladder 112 can also provide cooling to the compression site when the inflatable bladder 112 is at least partially inflated. In accordance therewith, the inflatable bladder 112 may be configured to hold a coolant solution that can assume a temperature that facilitates an absorption of heat from the compression site that results in cooling of said site. The valve 108 may be configured to facilitate injection of the coolant solution into the interior, and also withdrawal and replacement of the coolant solution. The valve 108 may be further configured for fluid communication with a delivery device or other source of coolant solution, as further described below.

The compression member 102 may include one or more features that indicate the current temperature of the bladder 112 and/or coolant solution, and thereby indicate the state of cooling provided to the compression site. In some embodiments, as illustrated in FIGS. 1A-1C, the compression member 102 may comprise a temperature indicator 110 configured to indicate the temperature of the coolant solution. The temperature indicator 110 may be situated so as to be in contact with the solution. For example, the temperature indicator 110 may be located within the interior of the inflatable bladder 112. The temperature indicator 110 may be unattached to the bladder so that the indicator can freely float on or in the coolant solution. In other embodiments, the temperature indicator 110 may be in direct contact with a surface of the inflatable bladder 112. For example, the temperature indicator 110 can be adhered to a surface of the inflatable bladder 112, either an outside surface or a surface of the bladder interior. In another example, the temperature indicator 110 may be integrated with the bladder itself, such as the bladder material or, if present, the top plate 114. For instance, the temperature indicator 110 can be integrated within the top plate 114 material such that a portion or all of the top plate 114 is considered the temperature indicator 110. Preferably, the temperature indicator 110 is situated so that it is visible in an external view of the compression device. For example, as shown in FIGS. 1A-1D the temperature indicator 110 may be visible through the window 116. It is also preferred that the temperature indicator 110 has a size and shape so that the temperature indicator 110 does not prevent visual observation of the compression site. In further embodiments, the temperature indicator 110 is integrated with the bladder or top plate 114 such that it is transparent or translucent to facilitate visible observation of the compression site.

In various embodiments, the temperature indicator may comprise a thermochromic feature in which the current temperature is indicated by the indicator's color or a change in temperature is indicated by a transition from one color to another color. In some embodiments, the temperature indicator is configured to indicate at least two temperature-related states of the coolant solution. Two such states may respectively be lower and higher than a target temperature. In some cases the target temperature may signify a transition point between a cooling state and a non-cooling state. A cooling state may comprise coolant solution temperatures that are sufficiently lower than that of the compression site so that heat will tend to move from the compression site into the coolant solution. Accordingly, the non-cooling state can comprise coolant solution temperatures at or above the point of thermoneutrality between the coolant solution and the compression site. A thermochromic indicator so configured may be able to assume a color in response to one of these temperature ranges and thereby indicate the corresponding state. The other state may be indicated by the absence of that color. For example, a thermochromic temperature indicator may assume a color to signify a cooling state in response to coolant solution temperatures at or below a target temperature of about 85° F., and may likewise not display said color when coolant solution temperature exceeds said target temperature.

In some embodiments, the thermochromic indicator may be further configured to assume at least two colors, wherein the indicator assumes a first color in response to one state and a second color in response to the other state. In a thermochromic temperature indicator capable of assuming more than two colors, the additional colors may reflect additional states or transitions between states. For example, a first color may indicate coolant solution temperatures that are effective for achieving hemostasis of the compression site, a second color may indicate coolant solution temperatures that are ineffective for this purpose, and a third color may indicate an intermediate range of temperatures over which limited cooling will occur but some further action (e.g., replacing the coolant solution or compression device) is needed to avoid loss of hemostasis.

In some embodiments the temperature indicator may include other features that indicate one or more temperature states. Nonlimiting examples include text, numbers, symbols, a scale bar, and a geometric pattern. In the case of text, numbers, and symbols, a single one of any of these may be used to indicate a temperature, or a plurality such as a series of numbers may be used to indicate multiple temperatures or a temperature range.

FIG. 1D is a top view of the compression device 100 of FIGS. 1A-1C providing a more complete view of the securement system 104. As described above, the securement system 104 may comprise one or more straps 106 configured to wrap around a portion of the patient and attach to itself or each other. In such embodiments, the straps 106 may comprise any suitable releasable securement mechanism, such as a hook-and-loop or hook-and-hook fastening mechanism, pressure sensitive adhesives, buckles, magnets, snaps, clasps, etc., all of which are contemplated to be within the scope of this disclosure. As shown in FIG. 1D, the plurality of straps 106 are collectively referred to with the reference numeral 106, while the four individual straps of the illustrated embodiment are designated with reference numerals 106a, 106b, 106c, and 106d. Disclosure regarding the size, shape, design, or other structure of any individual strap of the plurality of straps 106 may be applied to any other individual strap.

Each strap of the plurality of straps 106 may have the same length or the straps 106 may be of different lengths. In some embodiments, the length of one or more straps may be sufficient to extend around or partially around the torso of the patient. Each strap 106 may comprise a free end and may be coupled to the compression member 102 at a fixed end. The straps 106 may be generally elongate in shape, i.e., having a greater longitudinal length than width. The width may vary along the length of the strap 106. For example, in the illustrated embodiment, the width is narrower in a middle portion and wider at both the free end and the fixed end of each strap 106. Other shapes and designs of the straps 106 are likewise within the scope of this disclosure.

The plurality of straps 106 may be coupled to the compression member 102 such that each strap 106a, 106b, 106c, and 106d may pivot or rotate around the compression member 102. In other words, the straps 106 may be configured such that they may extend away from the compression member 102 at an adjustable direction or angle.

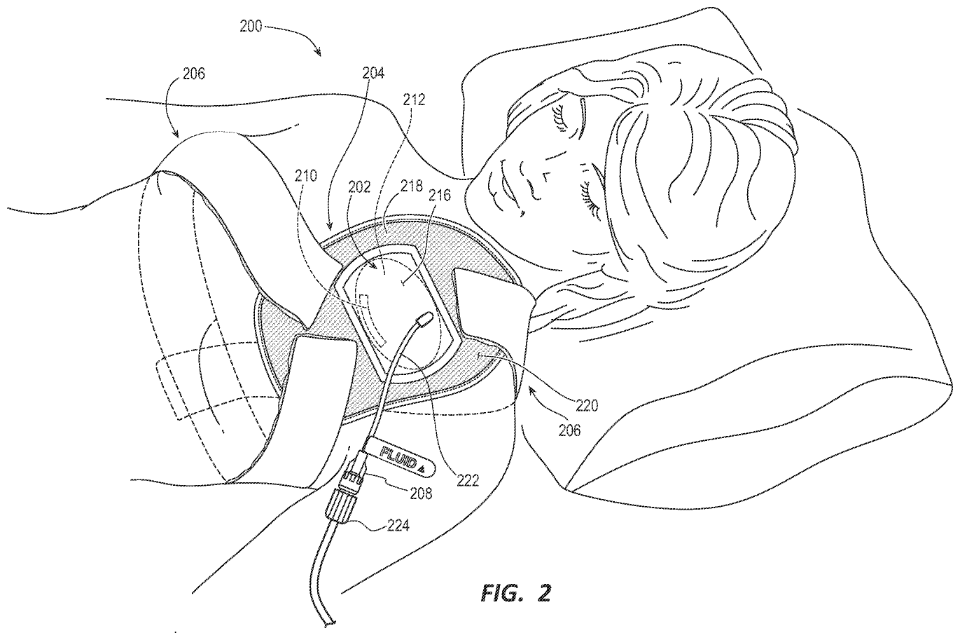

FIG. 2 is an illustration of an endothermic compression device 200 in use on a patient according to another embodiment. The endothermic compression device 200 resembles the compression device 100 described above in certain respects. Accordingly, like features and/or components of the compression device 200 are designated with like reference numerals, with the leading digits incremented to “2.” Relevant disclosure set forth above regarding similarly identified features and/or components of the compression member 202 thus may not be repeated hereafter. As shown, the compression device 200 can comprise a securement system 204 which can in turn comprise a collar 218 coupled to the compression member 202. In some embodiments, the collar 218 may be formed of a flexible material. The securement system 204 further comprises one or more straps 206 that may be selectively coupleable to the collar 218 and each other. The collar 218 and straps 206 may comprise any suitable releasable securement mechanism 220, such as a hook-and-loop or hook-and-hook fastening mechanism, pressure sensitive adhesives, buttons, buckles, magnets, snaps, clasps, etc. all of which are contemplated to be within the scope of this disclosure.

The compression device 200 may comprise a tube 222 coupled to the compression member 202 such that the tube 222 is in fluid communication with the inflatable bladder 212 at one end. The tube 222 may be coupled to the compression member 202 at a location toward an outer edge of the inflatable bladder 212 so as to not obstruct visibility through the window 216. A valve 208 may be coupled to the other end of the tube 222 so that the valve 208 is in fluid communication with the inflatable bladder 212. The tube 222 may provide flexibility between the valve 208 and the bladder thus providing for easier connection of an inflation fluid source 224 to the inflatable bladder 212. As shown, the compression member 202 may also comprise a temperature indicator 210 as described above.

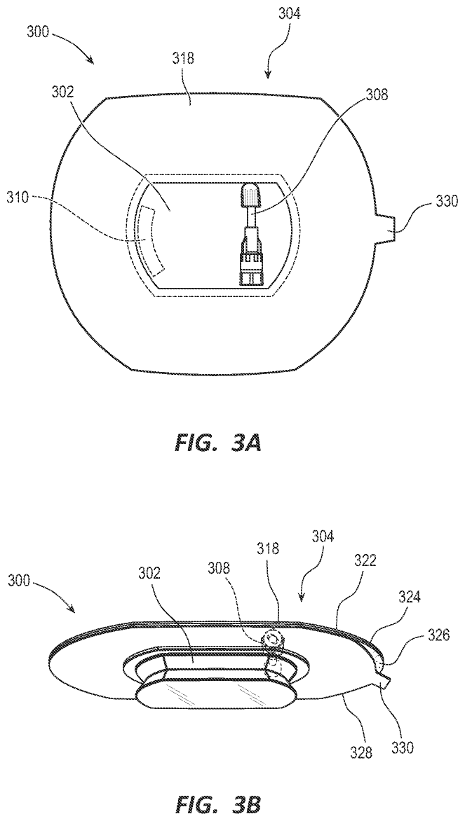

FIG. 3A and FIG. 3B are an illustration of another endothermic compression device 300. The inflatable compression device 300 resembles the compression device 200 described above in certain respects. Accordingly, like features and/or components of the compression device 300 are designated with like reference numerals, with the leading digits incremented to “3.” Relevant disclosure set forth above regarding similarly identified features and/or components of the compression device 300 thus may not be repeated hereafter.

As shown in the top view in FIG. 3A, the compression device 300 may comprise a securement system 304 that in turn comprises a collar 318 coupled to the compression member 302 comprising a temperature indicator 310. As illustrated in the view in FIG. 3B, the collar 318 may comprise a top layer 322, a bottom layer 324, an adhesive layer 326, and a release liner 328. The top layer 322 and bottom layer 324 can be formed of a suitable flexible material. The adhesive layer 326 can cover a bottom surface of the bottom layer 324. The adhesive layer 326 may comprise a pressure sensitive adhesive. The release liner 328 can be releasably coupled to the adhesive layer 326 to protect the adhesive layer 326 until the compression device is ready to use. A tab 330 can extend from any the release liner 328 to facilitate gripping of the release liner 328 for removal to expose the adhesive layer 326. When the adhesive layer 326 is exposed, the compression device 300 can be adhered to a patient's skin adjacent a surgical or incision site. In some embodiments, the top layer 322 and/or the bottom layer 324 may include a tab.

FIG. 3B shows the compression member 302 with the inflatable bladder (not visible) in a state of partial inflation. As shown, the compression member 302 may be configured to extend downward upon inflation of the inflatable bladder. In various embodiments described herein, the inflatable bladder may be disposed on the bottom surface of the compression member so that upward expansion of the inflatable bladder is limited or controlled. For example, where a top plate is included, the top plate limits or controls upward expansion of the inflatable bladder. The inflatable bladder may be configured to be in contact with a patient's skin and provide compression to the compression site. The inflatable bladder may be configured to be inflatable and deflatable by the passage of fluid through the valve 308. Displacement of fluid into or out of the inflatable bladder may thus inflate or deflate the inflatable bladder. Thus, the inflatable bladder may be configured to contain an inflation fluid, over a period of time, such as the desired time of compression and/or cooling of a compression site. In some embodiments, an internal fluid pressure within the inflatable bladder may correlate to the compressive pressure or level of compression applied to a patient. The inflatable bladder may be configured to provide the desired compression and/or cooling while only partially inflated.

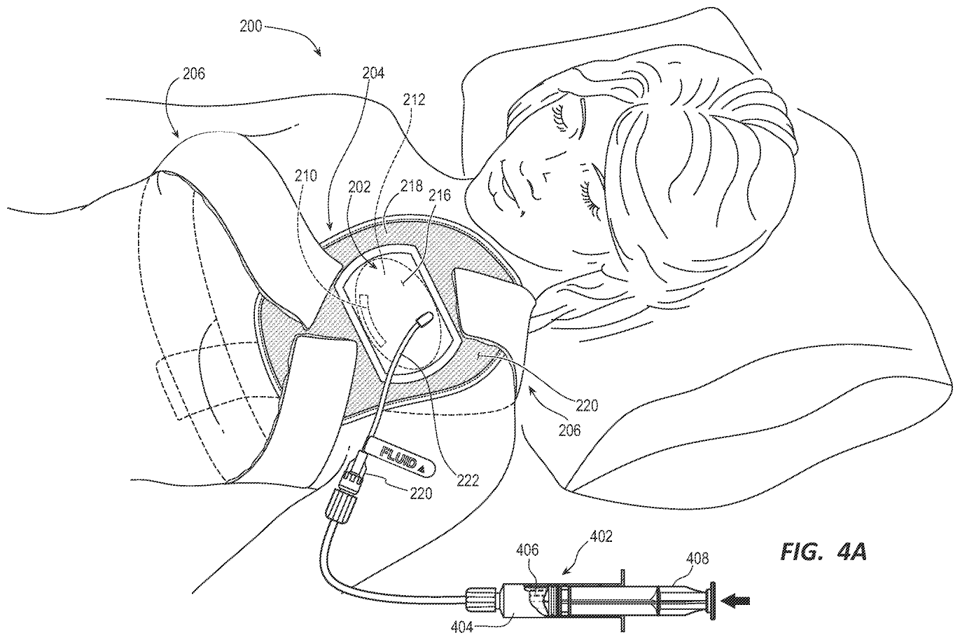

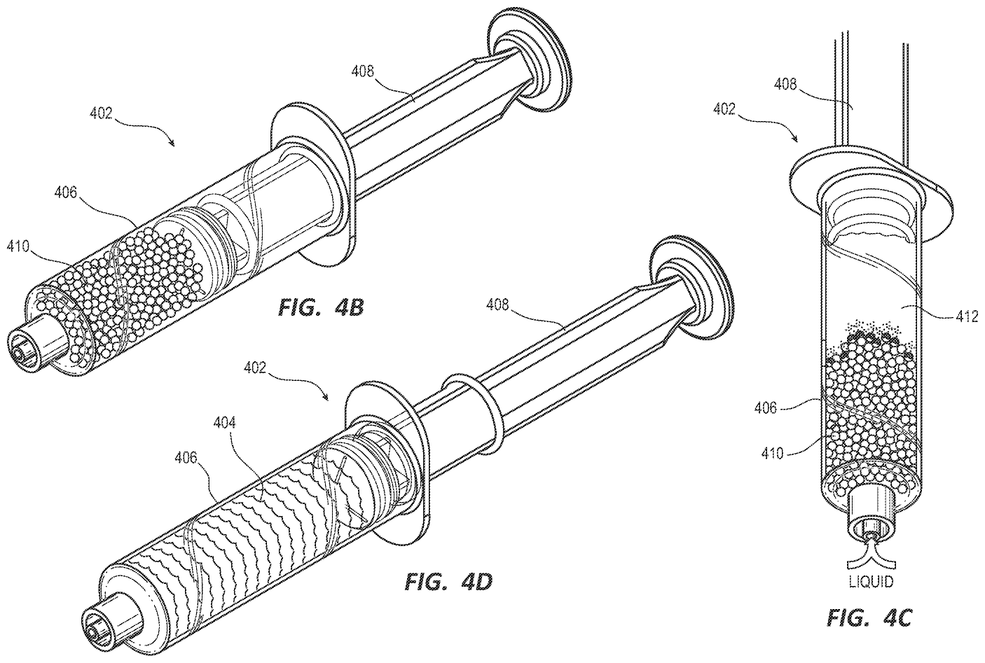

In some embodiments, a system for applying cooling and compression to a site on a body region of a subject provides for using a coolant solution in conjunction with an endothermic compression device. As illustrated in FIG. 4A, such a system 400 may comprise a compression device according to the above description (here compression device 200 of FIG. 2 is shown) and may further comprise a coolant delivery device 402 for delivering coolant solution 404 into the compression member 202. The coolant delivery device 402 may include a chamber 406 configured for holding a volume of coolant solution 404 that will provide a level of inflation of the inflatable bladder 212. The coolant delivery device 402 may also be configured to make a fluid connection with the inflatable bladder 212 e.g., through a valve 208, and include an expeller 408 for expelling coolant solution 404 from the chamber 406 into the inflatable bladder 212 through the fluid connection. In some embodiments as illustrated in FIG. 4A, the coolant delivery device 402 is a syringe, where the syringe barrel defines the chamber 406 and the plunger serves as the expeller 408.

In some cases, effective establishment or maintenance of hemostasis at a surgical site may depend upon the ability to initiate compression and cooling promptly after attaching the compression device to the patient. In some embodiments, a system for applying cooling and compression to a site on a body region of a subject may comprise a compression device, a coolant delivery device, and material(s) for making a coolant solution. The material(s) can comprise an endothermic material, that is, a material that is soluble in a liquid and lowers the temperature of the liquid as it dissolves. The endothermic material may be a solid material. In some embodiments, the coolant delivery device may be pre-loaded with a solid endothermic material from which a coolant solution may be made as needed. In some embodiments, the endothermic material may be pre-loaded in the inflatable bladder.

FIGS. 4B-4D are close-up views of the coolant delivery device 402 of FIG. 4A in which this function is illustrated further. As shown in FIG. 4B, the chamber 406 may be configured to contain an amount of solid endothermic material 410. A number of solid forms are encompassed by the present disclosure, including pellets, particles, powder, flakes and crystals. Such solid forms can also be dried or dehydrated. In an aspect, the solid form used can be selected to facilitate effective loading and retention in the chamber 406. In another aspect, the solid form can be selected to facilitate dissolution and particularly a rate of dissolution. In some cases, the solid form may be determined in part by the physical and chemical properties of the compound(s) constituting the endothermic material. In some embodiments, the endothermic material may be provided in pellet form, or more particularly in prill form. Prills can be particularly suited for combining a plurality of substances into a single granular form, and various prilling methods allow a significant degree of control over average prill size.

The endothermic material may be selected to produce a coolant solution having a desired temperature. In some embodiments, the coolant solution achieves a temperature that is therapeutically effective in achieving or maintaining hemostasis in the compression site, either alone or in combination with compression. The therapeutically effective temperature may depend in part upon the nature and location of the compression site. For example, a surgical site on the torso, such as a pocket for a pacemaker or defibrillator, may involve multiple smaller blood vessels, while other sites (e.g., a radial artery puncture site, or a femoral artery puncture site) may involve one or a few larger vessels. As such, different combinations of pressure and temperature may be indicated for these two types of compression sites. The cooling temperature is preferably at or near the lowest temperature that does not present a risk of injury to tissues in the compression site. For example, it is preferred that the coolant solution does not freeze upon formation so as to avoid tissue injury and also to preserve a degree of flexibility of the inflatable bladder. The coolant solution may also be configured so as not to reach temperatures below the freezing point of water. In some embodiments, the coolant solution assumes a temperature upon formation of about 33° F. to about 55° F., or more particularly about 35° F. to about 45° F. In some embodiments, the coolant solution remains within a temperature range of about 33° F. to about 65° F. for about 15 minutes to about 120 minutes, or about 15 minutes to about 60 minutes. In some embodiments, the temperature persists with this range for about 20 minutes to about 40 minutes.

The temperature of the coolant solution upon formation can be based on a number of parameters, including the heat of solution of the endothermic material, the selection and relative amounts of component compounds of the endothermic material, the particular liquid used as a solvent, and the rate of dissolution of the endothermic material. Any or all of these parameters may be selected and adjusted to determine the cooling properties of the coolant solution. In some embodiments, pellet or particle size may be selected to provide a particular rate of dissolution. The material may have a distribution of pellet or particle sizes having a selected mean pellet or particle size. And in some instances, the material may be present in a form in which substantially all pellets or particles are of a selected size.

Endothermic materials for use in accordance with the present disclosure may comprise one or more of ammonium nitrate, silver nitrate, urea, biuret, ammonium chloride, potassium nitrate, ammonium perchlorate, potassium chlorate, potassium perchlorate, potassium chloride, calcium ammonium nitrate, calcium nitrate tetrahydrate. In an embodiment, the endothermic material comprises at least about 55 wt % urea.

The endothermic material may further comprise one or more additives to confer desired physical and/or chemical properties. In some embodiments, the endothermic material further includes one or more dissolution accelerants including, but not limited to, potassium chloride. In particular embodiments, the endothermic material comprises a mixture of urea and potassium chloride. In an embodiment, the endothermic material comprises about 70 wt % to about 90 wt % urea and about 10 wt % to about 30 wt % potassium chloride. In some embodiments, the endothermic material includes one or more anticaking agents. Anticaking agents that may be used include, but are not limited to, calcium silicate, magnesium hydroxide carbonate, sodium aluminosilicate, sodium ferrocyanide and potassium ferrocyanide. In another embodiment, the endothermic material includes an additive such as silica gel.

As shown in FIG. 4C, the chamber 406 may also be configured to provide for combining the endothermic material 410 contained therein with a suitable liquid solvent 412 to form a coolant solution within the coolant delivery device. In some embodiments, the liquid solvent 412 may be an aqueous liquid e.g., water or a saline solution. The coolant delivery device 402, and more particularly the chamber 406, may be configured to receive a volume of the liquid 412 that will combine with the endothermic material 410 to produce a coolant solution 404 having a particular concentration of endothermic material 410. In some embodiments, the coolant delivery device can combine an amount of endothermic material 410 comprising urea with a volume of liquid solvent to produce a coolant solution comprising about 30 wt % to about 50 wt % urea. When the coolant delivery device is a syringe as in the embodiment shown in FIGS. 4A-4D, this may be accomplished by placing the barrel in fluid communication with a source of the liquid and withdrawing the plunger 408.

As shown in FIG. 4C and FIG. 4D, the endothermic material 410 dissolves in the liquid solvent 412 to form the coolant solution 404. The chamber 406 may have a volume that accommodates the endothermic material 410 and liquid solvent 412 and further includes a volume of empty space to allow for shaking or other agitation of the contents to accelerate coolant solution formation. If desired, a temperature indicator can also be disposed on the chamber 406 to indicate the temperature of the coolant solution 404.

Some embodiments of an endothermic compression device may employ other means of cooling a compression site to which the device is applied. In some embodiments, the compression member may include a cooling element that provides cooling in conjunction with the inflatable bladder. In certain embodiments, the compression member includes a thermoelectric cooling element, also termed a Peltier cooler. A Peltier cooler is a solid-state active heat pump in which heat is transferred from one side to the other when current is applied to the cooler. The amount of heat transferred can be linearly related to the amount of current powering the Peltier cooler. Through the use of two plates on opposite sides of the cooler, the Peltier cooler can transfer the heat from one plate (the “cold surface”) to the other (the “hot surface”), whereby the cold surface can function as a cooling surface. It should be understood that the modifiers “hot” and “cold” as used herein in reference to these two surfaces are intended to represent relative temperatures at each end of a temperature gradient, and should not be construed as referring to specific temperature value unless expressly stated otherwise.

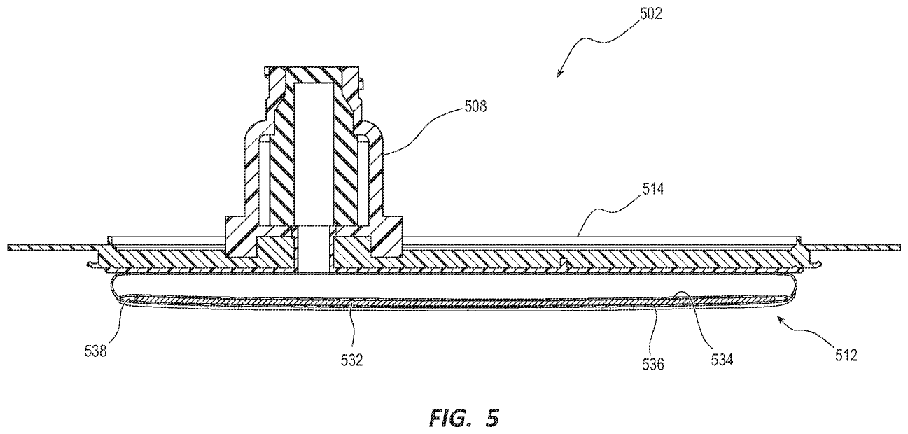

An exemplary embodiment is shown in cross-section view FIG. 5. A compression member 502 can comprise a top plate 514, an inflatable bladder 512 and a Peltier cooler 532. The Peltier cooler 532 can be activated by applying voltage to the cooler via electrical leads or contacts (not shown) to cause current to flow through the cooler's semiconductor array. This produces a temperature gradient, resulting in the cooler having a hot surface 534 and a cold surface 536. The Peltier cooler may be situated within the compression member 502 so that the cold surface 536 is adjacent a compression site—and therefore thermally coupled to said site—when the compression member is applied thereto. In an aspect, the inflatable bladder 512 can function as a heat sink for the hot surface 534 of the Peltier cooler 532, where the bladder can be inflated via the valve 508 with a fluid that absorbs heat from the hot surface. For example, as shown in FIG. 5, the Peltier cooler 532 can be situated so that the cold surface 536 is thermally coupled to the compression site and thereby cools the site, while the hot surface 534 is thermally coupled to the inflatable bladder 512.

The Peltier cooler 532 can be located within a pocket 538 situated on the underside of the compression member 502, for example formed in the material of the inflatable bladder 512. In another aspect, the Peltier cooler 532 can have a rigid structure that facilitates compression of a surgical site. For example, inflation of the inflatable bladder 512 can exert a downward force onto the Peltier cooler 532, which is in turn translated into a compressive force applied to the compression site over the surface area of the Peltier cooler 532. In certain embodiments, the Peltier cooler 532 can be removable from the pocket 538 such that it may be sanitized and/or reused.

The Peltier cooler 532 may be configured to provide cooling within a selected temperature range. The temperature range provided can depend on physical characteristics of the Peltier cooler 532, such as the dimensions and materials of the plates; and on the structure of its semiconductor array. The temperature range can also depend on the voltage applied to the cooler. In some embodiments, the Peltier cooler 532 can be activated to assume temperature at the cold surface 536 of about 33° F. to about 55° F., or more particularly about 35° F. to about 45° F. In some embodiments, a temperature indicator is functionally connected to the cold surface so as to indicate the temperature of the cold surface.

In some embodiments, the Peltier cooler is electrically connected to a control circuit configured for activation of the Peltier cooler by connecting the cooler to a voltage source. The control circuit can be further configured to keep the temperature of the cold surface of the cooler within a selected temperature range e.g., by controlling the timing and magnitude of the activating voltage. In some embodiments, the voltage source is a battery.

Various methods of use and treatment are within the scope of this disclosure. While certain examples of methods of treatment are described herein, methods within the scope of this disclosure include any subset of the steps recited and re-ordering of the steps as described.

An exemplary method of use of an endothermic compression device to apply compression with cooling to a compression site may comprise one or more of the following steps or processes. A practitioner may place the compression member on a body region and position the compression member over a compression site identified in said body region. The practitioner may view the compression site through the window while positioning and aligning the compression member. The practitioner may then secure the compression device to the body region with the securement system. The practitioner may then inflate the inflatable bladder with one or more inflation fluids. The inflatable bladder may be initially uninflated, partially inflated, or substantially fully inflated, or the inflatable bladder may initially contain a vacuum.

In various embodiments, inflation may be accomplished at least in part by injecting a volume of coolant solution into the interior of the bladder to provide cooling to the compression site. In some embodiments, inflation can comprise using a coolant delivery device such as described above, where the chamber of said device contains coolant solution. In an embodiment, the coolant delivery device is a syringe with a volume of coolant solution contained in the barrel. More particularly, the practitioner may fluidly connect the coolant delivery device to the inflatable bladder, e.g., via a valve if the bladder is so equipped, and inject a volume of the coolant solution into the inflatable bladder. In some embodiments, the procedure further comprises making a coolant solution by providing a coolant delivery device containing an amount of endothermic material such as described above, and introducing a volume of liquid solvent into the coolant delivery device to combine with the endothermic material. In an embodiment, the coolant delivery device is a syringe with an amount of endothermic material contained in the barrel. Liquid solvent can be added to the endothermic material by withdrawing the syringe plunger while the barrel is in fluid contact with a source of said solvent.

The practitioner may use an endothermic compression device as described herein to provide cooling therapy comprising a temperature or range of temperatures for a period of time. In accordance with some embodiments, this may be accomplished by at least partially inflating the bladder with a coolant solution having such a temperature and leaving the compression device in place for the desired therapy duration or while the coolant solution temperature remains within the target range. The temperature or range of temperatures may be selected to be therapeutically effective in achieving or maintaining hemostasis in the compression site, either alone or in combination with compression. Therapeutically effective temperatures may depend in part on the nature and location of the compression site. In some embodiments, cooling is provided by maintaining the coolant solution in the inflatable bladder within a temperature range of about 33° F. to about 65° F. for about 15 minutes to about 120 minutes, or about 15 minutes to about 60 minutes. In some embodiments, the temperature range is about 40° F. to about 60° F. In some embodiments, the temperature range is maintained for about 20 minutes to about 40 minutes.

The practitioner may ascertain the coolant solution temperature state by observing the temperature indicator, and may monitor the temperature state as the coolant solution gradually warms. If the temperature indicator indicates that the coolant solution is warming such that its temperature may exceed the desired temperature range before the desired time has expired, the practitioner may extend the cooling time by withdrawing the coolant solution from the bladder and replacing it with a quantity of coolant solution, e.g., freshly made coolant solution, having a temperature in the desired temperature range.

In accordance with some embodiments, providing cooling therapy comprising a temperature or range of temperatures for a period of time can comprise using an endothermic compression device having a compression member equipped with a thermoelectric cooling element such as a Peltier cooler. This method can comprise activating the Peltier cooler so as to initiate cooling of its cold side, for example, by applying a voltage to the Peltier cooler via electrical connections. In some embodiments, the Peltier cooler is activated via a control circuit configured to maintain cooling at or near the temperature or within the range of temperatures. For example, cooling may be maintained within a temperature range of about 33° F. to about 65° F. for about 15 minutes to about 120 minutes, or about 15 minutes to about 60 minutes. In some embodiments, the temperature range is about 40° F. to about 60° F. In some embodiments, the temperature range is maintained for about 20 minutes to about 40 minutes. In some embodiments, maintaining cooling temperature may comprise adding to or replacing a fluid in the inflatable bladder so as preserve the bladder's function as a heat sink for the hot surface.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment. Similarly, it should be appreciated by one of skill in the art with the benefit of this disclosure that in the above description of embodiments, various features are sometimes grouped together in a single embodiment, figure, or description thereof for the purpose of streamlining the disclosure.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. An endothermic compression device, comprising:
a compression member configured to apply compression to a compression site located in a body region of a patient, the compression member comprising:
an inflatable bladder having a fluid-tight interior configured to hold a volume of a coolant solution;
a valve in fluid communication with the interior; and
a temperature indicator configured to indicate one or more temperature states of the coolant solution; and
a securement system configured to secure the compression member to the body region of the patient.

2. The endothermic compression device of claim 1, wherein the temperature indicator comprises a thermochromic element configured to display a color to indicate one of the one or more temperature states.

3. The endothermic compression device of claim 1, wherein the one or more temperature states include a cooling state comprising coolant solution temperatures at or below a target temperature.

4. The endothermic compression device of claim 1, wherein the securement system comprises a collar coupled to the compression member and one or more straps coupleable to the collar.

5. The endothermic compression device of claim 1, wherein the compression member comprises a window providing visibility of the compression site through the compression member.

6. A system for applying cooling and compression to a site on a body region of a subject, comprising:
an endothermic compression device according to claim 1;
an amount of a solid endothermic material;
a coolant delivery device comprising a chamber configured for combining the endothermic material with an aqueous liquid to form the coolant solution and further configured to fluidly connect with the inflatable bladder and deliver the coolant solution to the interior.

7. The system of claim 6, wherein the endothermic material comprises one or more of ammonium nitrate, silver nitrate, urea, biuret, ammonium chloride, potassium nitrate, ammonium perchlorate, potassium chlorate, potassium perchlorate, potassium chloride, silica gel, calcium ammonium nitrate, and calcium nitrate tetrahydrate.

8. The system of claim 7, wherein the endothermic material further comprises at least one of a dissolution accelerant or an anticaking agent.

9. The system of claim 8, wherein the endothermic material comprises about 70 wt % to about 90 wt % urea and about 10 wt % to about 30 wt % potassium chloride.

10. The system of claim 6, wherein the coolant solution assumes a temperature upon formation of about 33° F. to about 55° F.

11. A method of applying compression with cooling to a compression site on a body region of a subject, comprising:
providing an endothermic compression device according to claim 1;
placing the compression member on the body region so that the inflatable bladder is situated directly over the compression site;
securing the endothermic compression device to the body region;
injecting a volume of the coolant solution through the valve into the interior of the inflatable bladder to provide cooling to the compression site at a temperature for a period of time.

12. The method of claim 11, wherein injecting the volume of the coolant solution further comprises:
providing a coolant delivery device having a chamber containing an endothermic material, wherein the coolant delivery device is configured to fluidly connect with the inflatable bladder through the valve;
forming the coolant solution by adding an aqueous liquid to the chamber to combine with the endothermic material;
fluidly connecting the coolant delivery device to the valve; and
injecting the coolant solution through the valve into the interior of the inflatable bladder.

13. The method of claim 11, further comprising maintaining cooling within a temperature range for the period of time by replacing the volume of coolant solution with a second volume of coolant solution having a temperature within the temperature range.

14. The method of claim 11, wherein the period of time is about 15 minutes to about 120 minutes.

15. The method of claim 13, wherein the temperature range is about 33° F. to about 65° F.

16. An endothermic compression device, comprising:
a compression member configured to apply compression to a compression site located in a body region of a patient, the compression member comprising:
an inflatable bladder having a fluid-tight interior;
a Peltier cooler having a hot surface and a cold surface, wherein the hot surface is thermally coupled to the inflatable bladder and the cold surface is situated to provide cooling to the compression site; and
a securement system configured to secure the compression member to the body region of the patient.

17. The endothermic compression device of claim 16, further comprising a control circuit configured to electrically couple the Peltier cooler to a voltage source.

18. The endothermic compression device of claim 17, wherein the control circuit is configured to control a voltage applied to the Peltier cooler to maintain cooling by the cold surface within a selected temperature range.

19. The endothermic compression device of claim 16, wherein the inflatable bladder is at least partially inflated with a fluid capable of absorbing heat from the hot surface.

20. The endothermic compression device of claim 16, comprising a temperature indicator configured to indicate a temperature of the cold surface.
